(19) United States
(12) Patent Application Publication
(10) Pub. No.: US 2025/0140361 A1
(43) Pub. Date: May 1, 2025

(54) METHOD FOR DETERMINING QTc OF A SUBJECT EXPECTED FROM ADMINISTRATION OF A SELECTED DRUG

(71) Applicant: AltaThera Pharmaceuticals LLC, Chicago, IL (US)

(72) Inventors: Brandon Ira Kashfian, Chicago, IL (US); Nico Punt, Chicago, IL (US); Adam Strickberger, Chicago, IL (US); Sander Vinks, Chicago, IL (US); Jan Wald, Chicago, IL (US)

(21) Appl. No.: 19/009,774

(22) Filed: Jan. 3, 2025

Related U.S. Application Data
(63) Continuation-in-part of application No. 18/631,538, filed on Apr. 10, 2024.
(63) Continuation-in-part of application No. 18/324,703, filed on May 26, 2023.
(63) Continuation-in-part of application No. 18/323,337, filed on May 24, 2023.
(63) Continuation-in-part of application No. 18/321,220, filed on May 22, 2023.
(63) Continuation-in-part of application No. 18/315,790, filed on May 11, 2023.
(63) Continuation-in-part of application No. 18/306,660, filed on Apr. 25, 2023.
(63) Continuation-in-part of application No. 18/304,196, filed on Apr. 20, 2023.
(63) Continuation-in-part of application No. 18/135,467, filed on Apr. 17, 2023.
(63) Continuation-in-part of application No. 18/126,561, filed on Mar. 27, 2023.
(63) Continuation-in-part of application No. 18/121,980, filed on Mar. 15, 2023.
(63) Continuation-in-part of application No. 18/107,785, filed on Feb. 9, 2023.
(60) Provisional application No. 63/741,691, filed on Jan. 3, 2025.
(60) Provisional application No. 63/640,469, filed on Apr. 30, 2024.
(60) Provisional application No. 63/640,412, filed on Apr. 30, 2024.
(60) Provisional application No. 63/565,774, filed on Mar. 15, 2024.
(60) Provisional application No. 63/617,390, filed on Jan. 3, 2024.
(60) Provisional application No. 63/346,003, filed on May 26, 2022.
(60) Provisional application No. 63/345,068, filed on May 24, 2022.
(60) Provisional application No. 63/345,068, filed on May 24, 2022.
(60) Provisional application No. 63/344,154, filed on May 20, 2022.
(60) Provisional application No. 63/345,068, filed on May 24, 2022.
(60) Provisional application No. 63/344,154, filed on May 20, 2022.
(60) Provisional application No. 63/340,581, filed on May 11, 2022.
(60) Provisional application No. 63/345,068, filed on May 24, 2022.
(60) Provisional application No. 63/344,154, filed on May 20, 2022.
(60) Provisional application No. 63/340,581, filed on May 11, 2022.
(60) Provisional application No. 63/332,882, filed on Apr. 20, 2022.
(60) Provisional application No. 63/331,467, filed on Apr. 15, 2022.
(60) Provisional application No. 63/346,003, filed on May 26, 2022.
(60) Provisional application No. 63/332,882, filed on Apr. 20, 2022.
(60) Provisional application No. 63/323,836, filed on Mar. 25, 2022.
(60) Provisional application No. 63/332,882, filed on Apr. 20, 2022.
(60) Provisional application No. 63/331,467, filed on Apr. 15, 2022.
(60) Provisional application No. 63/334,267, filed on Apr. 25, 2022.

(51) Int. Cl. G16H 20/10; G16H 50/70

(52) U.S. Cl. CPC G16H 20/10; G16H 50/70

(57) ABSTRACT

Computer-implemented methods of predicting the QTc of a subject in response to administration of QTc prolonging drugs, especially those that are dose adjusted for renal function, such as antiarrhythmic drugs. Included is a method of administering an antiarrhythmic drug to a subject, comprising: providing as inputs into a treatment management application i) Baseline QTc and ii) at least a Creatinine Clearance, Weight, Age, Sex, or Heart Rate; the treatment management application providing as an output one or more predicted QTc or predicted QTc range for the subject expected from administration of a 1-5 hour IV dose, alone or followed by one or more oral doses, of an antiarrhythmic drug to the subject; and upon the one or more QTc or QTc range being within an acceptable range, administering the 1-5 hour IV dose to the subject optionally followed by administering one or more of the oral doses to the subject.

### CROSS-REFERENCE TO RELATED APPLICATIONS

This application claims priority to and the benefit of the filing date of U.S. Application Nos.:
63/741,691 filed Jan. 3, 2025;
63/640,469 filed Apr. 30, 2024;
63/640,412 filed Apr. 30, 2024;
Ser. No. 18/631,538 filed Apr. 10, 2024;
63/565,774 filed Mar. 15, 2024;
63/617,390, filed Jan. 3, 2024;
Ser. No. 18/324,703 filed May 26, 2023 and which claims priority to U.S. Application No. 63/346,003 filed May 26, 2022;
Ser. No. 18/323,337 filed May 24, 2023 and which claims priority to U.S. Application No. 63/345,068 filed May 24, 2022;
Ser. No. 18/321,220 filed May 22, 2023 and which claims priority to U.S. Application Nos. 63/345,068 filed May 24, 2022 and 63/344, 154 filed May 20, 2022;
Ser. No. 18/315,790 filed May 11, 2023 and which claims priority to U.S. Application Nos. 63/345,068 filed May 24, 2022, 63/344,154 filed May 20, 2022 and 63/340,581 filed May 11, 2022;
Ser. No. 18/306,660 filed Apr. 25, 2023 and which claims priority to U.S. Application Nos. 63/345,068 filed May 24, 2022, 63/344,154 filed May 20, 2022, 63/340,581 filed May 11, 2022 and 63/334,267 filed Apr. 25, 2022;
Ser. No. 18/304,196 filed Apr. 20, 2023 and which claims priority to U.S. Application No. 63/332,882 filed Apr. 20, 2022;
Ser. No. 18/135,467 filed Apr. 17, 2023 and which claims priority to U.S. Application Nos. 63/331,467 filed Apr. 15, 2022, 63/346,003 filed May 26, 2022 and 63/332,882 filed Apr. 20, 2022;
Ser. No. 18/126,561 filed Mar. 27, 2023 and which claims priority to U.S. Application Nos. 63/323,836 filed Mar. 25, 2022, 63/332,882 filed Apr. 20, 2022 and 63/331,467 filed Apr. 15, 2022;
Ser. No. 18/121,980 filed Mar. 15, 2023; and
Ser. No. 18/107,785 filed Feb. 9, 2023;
which applications are each hereby incorporated by reference herein in their entireties.

### FIELD OF THE INVENTION

The present disclosure is directed to the field of cardiovascular pharmaceutics, and more particularly to computer-implemented methods of determining/predicting QTc or a QTc range for a subject expected after administration of a selected drug, such as an antiarrhythmic drug, and/or determining appropriate dose amount(s), such as for IV and/or oral dosing, of the selected drug. Medical devices and systems capable of performing such methods are also included. In addition to antiarrhythmic drugs, the methods, systems and devices of the invention are applicable to any QTc prolonging drug, especially QTc prolonging drugs dose adjusted for renal function.

### BACKGROUND

Determining the appropriate dosing of antiarrhythmic drugs can be complex, difficult to understand or apply, and/or time consuming. In situations where a patient is having a cardiovascular crisis, practitioners need to act quickly and, thus, make decisions quickly. Dosing an antiarrhythmic drug, however, often involves simultaneous and careful consideration of a plethora of parameters. Typical dosing parameters include whether to administer the drug to a patient based on the patient's QT interval or QTc and/or the patient's capability of clearing the drug from their body (such as renal clearance as measured by creatinine clearance rate). Software, such as a mobile application or app, appropriately programmed, can be used to determine and/or confirm a practitioner's determination of whether to administer a drug to a particular patient based on one or more or all of the above parameters, and/or other relevant parameters. The systematic application of estimating QTc by a software app, when applied to the circumstances of a particular patient, can increase the safety of dosing/administering antiarrhythmic drugs, such as sotalol, dofetilide, amiodarone, ibutilide, dronedarone, procainamide, flecainide, or propafenone.

Sotalol is an antiarrhythmic drug with Class II (beta-adrenoreceptor blocking) and Class III (cardiac action potential duration prolongation) properties. Sotalol is indicated for the maintenance of normal sinus rhythm (delay in time to recurrence of atrial fibrillation/atrial flutter (AFIB/AFL)) in patients with symptomatic AFIB/AFL who are currently in sinus rhythm, and indicated for the treatment of life-threatening ventricular tachycardia.

Adult dosages for oral sotalol can include 80 mg, 120 mg, 160 mg, or 240 mg, while intravenous doses of sotalol can range from 20-240 mg.

Sotalol hydrochloride is a white, crystalline solid with a molecular weight of 308.8. It is hydrophilic, soluble in water, propylene glycol and ethanol, but is only slightly soluble in chloroform. Chemically, sotalol hydrochloride is d,1-N-[4-[1-hydroxy-2-[(1-methylethyl)amino]ethyl]phenyl]methane-sulfonamide monohydrochloride. The molecular formula is C₁₂ H₂₀ N₂ O₃S·HCl. Sotalol is represented by the following structural formula:

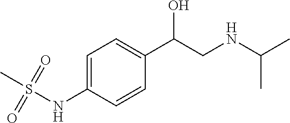 [SMILES: CC(C)NCC(O)c1ccc(NS(C)(=O)=O)cc1]

Sotalol can cause serious ventricular arrhythmias, primarily Torsade de Pointes (TdP) type ventricular tachycardia, a polymorphic ventricular tachycardia associated with QTc prolongation. QTc prolongation is directly related to the concentration of sotalol in the patient. As a result, the US FDA has mandated in-hospital QTc monitoring for at least three days upon initial sotalol hydrochloride loading and for dose escalation. Sotalol is currently approved in the US for oral administration (for example, under the brand name BETAPACE AF®, Bayer HealthCare Pharmaceuticals Inc.) and is approved for IV administration (AltaThera Pharmaceuticals LLC).

Ibutilide as an antiarrhythmic drug is characterized by predominantly Class III properties and is represented by the following structural formula:

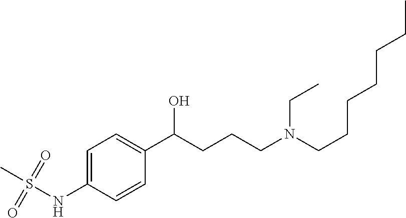 [SMILES: CCCCCCCN(CC)CCCC(O)c1ccc(NS(C)(=O)=O)cc1]

Ibutilide (a methanesulfonanilide derivative) is currently approved in the United States for intravenous administration (under the brand name CORVERT®, Pfizer Inc.) for the treatment of atrial fibrillation or atrial flutter. Patients with atrial arrhythmias of recent onset are more likely to respond to ibutilide. Typical adult IV doses of ibutilide are in the range of about 1-2 mg.

Another antiarrhythmic, dofetilide, is a Class III antiarrhythmic agent represented by the following structural formula:

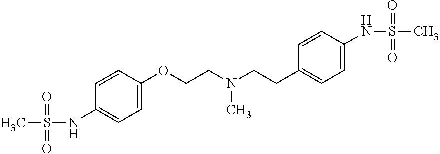

The mechanism of action for dofetilide is through blocking cardiac ion channels of the rapid component of the delayed rectifier potassium current Ikr. The agent, a sulfonamide, is approved to treat atrial fibrillation and atrial flutter. Dofetilide may additionally be useful for hospitalized people with atrial fibrillation cardioverted to normal sinus rhythm, without ventricular arrhythmias or various forms of blocks, in patients with normal kidney function. Dofetilide normalizes sinus rhythm by prolonging cardiac action potential duration and effective refractory period due to delayed repolarization without affecting conduction velocity. Dofetilide is currently approved in the United States for oral administration (under the brand name TIKOSYN®, Pfizer Inc.). Typical oral doses of dofetilide are in the range of 125-500 mcg (twice daily), while intravenous doses can be in the range of about 0.1 mcg/kg to 3 mcg/kg or more.

Amiodarone (for example, amiodarone hydrochloride: 2-butyl-3-benzafuranyl)[4-[2-(diethylamino)ethoxy]-3,5-diiodophenyl]-methanone hydrochloride) is also a Class III antiarrhythmic drug and is represented by the following structural formula:

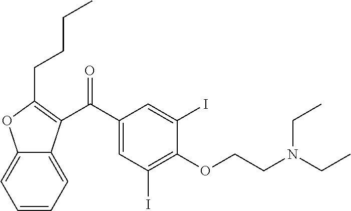 [SMILES: CCCCc1oc2ccccc2c1C(=O)c1cc(I)c(OCCN(CC)CC)c(I)c1]

Amiodarone is used for the treatment of life-threatening recurrent VF or life-threatening recurrent hemodynamically unstable VT and is usually used for patients who are intolerant to or who may not respond adequately to other antiarrhythmic drugs. It is currently approved in the United States for both oral and intravenous administration and is marketed under brand names including CORDARONE®, PACERONE®, and NEXTERONE®. Typical doses for oral amiodarone are in the range of 100-400 mg, with a loading dose of 800-1600 mg/day and a maintenance dose of 600-800 mg/day tapering off to 400 mg/day, while intravenous dosing of amiodarone is in the range of 150-540 mg.

Dronedarone (400 mg tablets, typically administered twice daily) is used for the treatment of paroxysmal or persistent atrial fibrillation or atrial flutter and can be used with patients who have experienced a recent AF/AFL episode who are in sinus rhythm or who will be cardioverted. Dronedarone is marketed under the brand name MULTAQ® (Sanofi Aventis) and has the following structural formula:

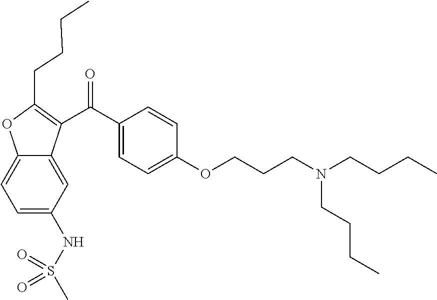 [SMILES: CCCCc1oc2ccc(NS(C)(=O)=O)cc2c1C(=O)c1ccc(OCCCN(CCCC)CCCC)cc1]

Flecainide is a Class IC drug used for the treatment of ventricular tachycardia, atrial fibrillation, atrial flutter, Wolff-Parkinson-White syndrome and paroxysmal supraventricular tachycardia and is represented by the following structural formula:

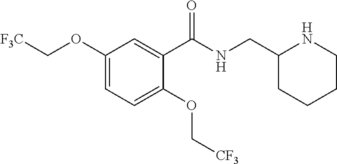 [SMILES: O=C(NCC1CCCCN1)c1cc(OCC(F)(F)F)ccc1OCC(F)(F)F]

Flecainide is typically administered to adults starting with 50-100 mg (twice a day) with the maximum recommended dose being 400 mg/day and is sold under the brand name TAMBOCOR® (3M Pharmaceuticals). Renal and liver adjustments may be needed in some cases.

Procainamide is typically administered in oral doses of 1500-2500 mg (twice daily) to adults and for intravenous doses in the range of 15-18 mg/kg or 100 mg, and up to a total of 1 gram/day. For patients with low renal or liver clearance abilities, adjustments are recommended. Procainamide is marketed under the brand names including PRONESTYL®, PRONESTYL-SR®, PROCAN SR®, and PROCANBID®.

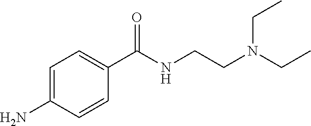 [SMILES: CCN(CC)CCNC(=O)c1ccc(N)cc1]

Propafenone (RHTHMOL®, RYTHMOL SR®) is used for the treatment of atrial fibrillation, atrial flutter, ventricular tachycardia and paroxysmal supraventricular tachycardia. A typical adult oral dose is in the range of about 150-425 mg, such as 150 mg at 8 hour intervals with a maximum dose of 800 mg/day. Adjustments, such as dose reduction, may be needed for patients with renal or liver clearance difficulty.

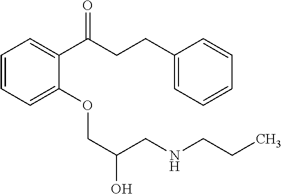 [SMILES: CCCNCC(O)COc1ccccc1C(=O)CCc1ccccc1]

More specific dosing information, mechanisms of action, contraindications, adverse effects, drug interactions, if any, renal and/or liver clearance thresholds, if applicable, etc. for various antiarrhythmic drugs can be found in the following product labels: (1) Amiodarone HCl injection for intravenous use [package insert], Lake Forest, IL: Hospira, Inc.; Initial U.S. Approval: 1995; (2) Cordarone® (amiodarone HCl) Tablets [package insert], Philadelphia, PA: Wyeth Pharmaceuticals Inc.; 2004; (3) TIKOSYN® (dofetilide) Capsules [package insert], NY, NY: Pfizer Inc.; 2014; (4) MULTAQ (dronedarone) tablets, for oral use [package insert], Bridgewater, NJ: Sanofi-Aventis U.S. LLC; 2020; (5) FLECAINIDE ACETATE TABLETS, USP [package insert], Jacksonville, FL: Ranbaxy Pharmaceuticals Inc.; 2003; (6) IBUTILIDE FUMARATE INJECTION [package insert], Morgantown, WV: Mylan Institutional LLC; 2020;(7) PROCAINAMIDE HYDROCHLORIDE Injection, USP [package insert], Lake Forest, IL: Hospira, Inc.; 2021; (8) RYTHMOL (propafenone hydrochloride tablets), for oral use [package insert], Research Triangle Park, NC: GlaxoSmithKline; 2018. Each of these product labels is hereby incorporated by reference herein in its entirety.

### SUMMARY OF THE INVENTION

Aspects of embodiments of the invention include:

Aspect 1, which is a system for treatment planning, comprising: i) a database of a number of subject profiles, each comprising a QTc or QTc range resulting from or expected to result from administration of a drug to the subject of the profile at a selected amount, optionally wherein the subject profiles are simulated subject profiles obtained from clinical trial simulations on a PK/PD model for the selected drug and/or are actual subject profiles comprising data obtained from actual subjects; ii) a processor capable of executing computer-executable instructions for determining an expected QTc or QTc range for a current actual subject, wherein the determining is based on the subject profiles and a Baseline QTc and Creatinine Clearance, Weight, Age, Sex, and/or Heart Rate of the current actual subject, which expected QTc or QTc range would be expected to result after administration of the drug to the current actual subject at the selected amount; iii) optionally comprising a display for visually displaying one or more of the subject's Baseline QTc, a threshold QTc (or allowable % change), an expected median QTc or QTc range expected to result from administration of the drug to the current actual subject at the selected amount, and/or a 95% confidence interval of the expected QTc or QTc range.

Aspect 2 is a treatment management application configured to perform a set of operations comprising: (i) reading, prompting input of and/or accepting the following as inputs: (a) a subject's Baseline QTc and one or more of the subject's Creatinine Clearance, Weight, Age, Sex, and/or Heart Rate, optionally before administration of a drug to the subject; (b) an amount of an oral or IV target dosage of the drug for the subject; and (ii) executing computer executable instructions to derive from a set of rules an expected QTc or QTc range for the subject based on profiles of other subjects and the subject's Baseline QTc and Creatinine Clearance, Weight, Age, Sex, and/or Heart Rate, which expected QTc or QTc range would be expected to result from administration of the drug to the subject at the selected amount; (iii) outputting the expected QTc or QTc range, optionally by way of a visual display of one or more of the subject's Baseline QTc, a threshold QTc (or allowable % change), an expected median QTc or QTc range expected to result from administration of the drug to the subject at the selected amount, and/or a 95% confidence interval of the expected QTc or QTc range.

Aspect 3 is the treatment management application of Aspect 2, wherein the profiles of other subjects comprise a simulated QTc or QTc range expected to result after administration of the drug to the subject of a simulation at the selected amount, optionally wherein the profiles of the other subjects are simulated subject profiles obtained from clinical trial simulations on a PK/PD model for the selected drug and/or are actual subject profiles comprising data obtained from other actual subjects.

Aspect 4 is a method of administering an antiarrhythmic drug to a patient, comprising: (a) providing patient-related inputs into a treatment management application comprising at least i) a patient's Baseline QTc and ii) one or more of a patient's Creatinine Clearance, Weight, Age, Sex, or Heart Rate; (b) the treatment management application providing as an output one or more QTc or QTc range for the patient expected from administration of a 1-5 hour IV dose, alone or followed by one or more oral doses, of an antiarrhythmic drug to the patient; and (c) in response to the one or more QTc or QTc range being within an acceptable range, administering the 1-5 hour IV dose of the antiarrhythmic drug to the patient, optionally followed by administering one or more of the oral doses of the antiarrhythmic drug to the patient.

Aspect 5 is any of Aspects 1-4, wherein the drug or antiarrhythmic drug is selected from sotalol, dofetilide, amiodarone, ibutilide, dronedarone, procainamide, flecainide, or propafenone.

Aspect 6 is any of Aspects 1-5, wherein the drug or antiarrhythmic drug is sotalol hydrochloride.

Aspect 7 is any of Aspects 1-6, wherein the administering comprises administering to the patient: a 1-hour IV loading dose of sotalol hydrochloride; and one or more oral doses of 80 mg, 120 mg, 160 mg or 240 mg sotalol hydrochloride.

Aspect 8 is any of Aspects 1-7, wherein: at least the patient's Baseline QTc and Creatinine Clearance are the patient-related inputs and a target oral dose for the patient is input into the treatment management application; and the amount and duration of the 1-5 hour IV dose are determined from a set of rules based on at least the patient's creatinine clearance and the target oral dose for the patient.

Aspect 9 is any of Aspects 1-8, wherein: the treatment management application comprises or is in operable communication with a database comprising a number of simulated and/or actual subject profiles, each of the subject profiles comprising a QTc or QTc range resulting from or expected to result from administration of the antiarrhythmic drug to the subject of the profile at a selected amount and duration; the output of the one or more QTc or QTc range expected for the patient is derived from a set of rules based on the simulated and/or actual subject profiles and the patient-related inputs.

Aspect 10 is any of Aspects 1-9, wherein: the treatment management application comprises or is in operable communication with a database comprising a number of simulated and/or actual subject profiles, each of the subject profiles comprising a QTc or QTc range resulting from or expected to result from administration of the antiarrhythmic drug to the subject of the profile at a selected amount and duration; the output of the one or more QTc or QTc range expected for the patient is derived from a set of rules based on the simulated and/or actual subject profiles and the patient-related inputs.

Aspect 11 is any of Aspects 1-10, wherein the treatment management application comprises or is in operable communication with a neural network trained on an initial set of simulated subject profiles and/or additional simulated and/or actual subject profiles, optionally updated in response to obtaining additional simulated subject profiles and/or actual subject profiles.

Aspect 12 is any of Aspects 1-11, wherein the treatment management application comprises or is in operable communication with a neural network trained on an initial set of simulated subject profiles and/or additional simulated and/or actual subject profiles, optionally updated in response to obtaining additional simulated subject profiles and/or actual subject profiles.

Aspect 13 is any of Aspects 1-12, wherein the output of the one or more QTc or QTc range expected for the patient is determined: performing stochastic simulations; using an object-oriented approach for model simulation in which each dose is represented by its closed form exponential representation; and/or using a plasma curve constructed by superposition; and/or using real-time Monte Carlo simulations.

Aspect 14 is any of Aspects 1-13, wherein the output of the one or more QTc or QTc range expected for the patient is determined: performing stochastic simulations; using an object-oriented approach for model simulation in which each dose is represented by its closed form exponential representation; and/or using a plasma curve constructed by superposition; and/or using real-time Monte Carlo simulations.

Aspect 15 is any of Aspects 1-14, wherein the administering comprises administering to the patient: a 1-hour IV loading dose of dofetilide; and one or more oral doses of 125 μg, 250 μg, or 500 μg dofetilide.

Aspect 16 is any of Aspects 1-15, wherein: at least the patient's Baseline QTc and Creatinine Clearance are the patient-related inputs and a target oral dose for the patient is input into the treatment management application; and the amount and duration of the 1-5 hour IV dose are determined from a set of rules based on at least the patient's creatinine clearance and the target oral dose for the patient.

Aspect 17 is any of Aspects 1-16, wherein: the treatment management application comprises or is in operable communication with a database comprising a number of simulated and/or actual subject profiles, each of the subject profiles comprising a QTc or QTc range resulting from or expected to result from administration of the antiarrhythmic drug to the subject of the profile at a selected amount and duration; the output of the one or more QTc or QTc range expected for the patient is derived from a set of rules based on the simulated and/or actual subject profiles and the patient-related inputs.

Aspect 18 is any of Aspects 1-17, wherein the treatment management application comprises or is in operable communication with a neural network trained on an initial set of simulated subject profiles and/or additional simulated and/or actual subject profiles, optionally updated in response to obtaining additional simulated subject profiles and/or actual subject profiles.

Aspect 19 is any of Aspects 1-18, wherein the output of the one or more QTc or QTc range expected for the patient is determined: performing stochastic simulations; using an object-oriented approach for model simulation in which each dose is represented by its closed form exponential representation; and/or using a plasma curve constructed by superposition; and/or using real-time Monte Carlo simulations.

Aspect 20 is a method, comprising: performing clinical trial simulations on a PK/PD model for a selected drug to obtain a number of simulated subject profiles, each comprising a simulated QTc range expected to result after administration of the drug at a selected amount to the subject of the simulation.

### BRIEF DESCRIPTION OF THE DRAWINGS

The accompanying drawings illustrate certain aspects of implementations of the present disclosure and should not be construed as limiting. Together with the written description the drawings serve to explain certain principles of the disclosure.

FIG. 1 is an illustration showing representative inputs and outputs for a dosing software according to embodiments of the invention.

FIG. 2 is a graph showing predicted QTc for a representative subject administered a 75 mg dose of IV sotalol, according to an embodiment of the invention.

FIG. 3 is an illustration showing software dosing recommendation and QTc prediction according to an embodiment of the invention, and although both are shown the software can be configured to provide either or both dosing recommendation and/or QTc prediction.

FIG. 4 is an illustration showing the introductory software screens including a warning screen for sotalol and the patient type selection screen, according to embodiments of the invention.

FIG. 5 is an illustration showing representative inputs and outputs for adult dosing for the entered creatinine clearance option, according to embodiments of the invention.

FIG. 6 is an illustration showing software screens for adult dosing for the calculated creatinine clearance option, according to embodiments of the invention.

FIG. 7 is an illustration showing representative software inputs and outputs for the QTc prediction option, according to embodiments of the invention.

FIG. 8 is an illustration showing representative software inputs and outputs for the pediatric dosing option, according to embodiments of the invention.

FIG. 9 is an illustration showing representative software inputs and outputs for the escalation option for adult dosing according to embodiments of the invention.

### DETAILED DESCRIPTION OF VARIOUS EMBODIMENTS OF THE INVENTION

The detailed description provided below is intended as a description of the present examples and is not intended to represent the only forms in which the present example may be constructed or utilized. The description sets forth the functions of the examples and the sequence of steps for constructing and operating the examples. However, the same or equivalent functions and sequences may be accomplished by different examples.

Described herein are methods of administering one or more antiarrhythmic drug, such as a Class I, Class II or Class III antiarrhythmic drug, such as sotalol, dofetilide, amiodarone, ibutilide, dronedarone, procainamide, flecainide, or propafenone, to a patient in need thereof in an amount effective for treating a cardiovascular condition of the patient, such as: atrial fibrillation (AFIB); atrial flutter (AF or AFL); ventricular tachycardia; hemodynamically stable or unstable ventricular tachycardia (VT); ventricular fibrillation (VF); paroxysmal supraventricular tachycardia; paroxysmal atrial fibrillation; heart failure; coronary artery disease; pulmonary artery hypertension; maintenance of normal sinus rhythm (delay in time to recurrence of atrial fibrillation/atrial flutter (AFIB/AFL)) for example in patients with symptomatic AFIB/AFL and/or who are currently in sinus rhythm, and/or indicated for the treatment of life-threatening ventricular tachycardia; and/or treating people, such as hospitalized people, with atrial fibrillation cardioverted to normal sinus rhythm, without ventricular arrhythmias or various forms of blocks, in patients with normal kidney function; treating life-threatening recurrent VF or life-threatening recurrent hemodynamically unstable VT; treating paroxysmal or persistent atrial fibrillation or atrial flutter, such as with patients who have experienced a recent AF/AFL episode who are in sinus rhythm or who will be cardioverted; and/or treating Wolff-Parkinson-White syndrome and/or paroxysmal supraventricular tachycardia.

The methods, systems and devices of the invention are applicable to any QTc prolonging drug, especially QTc prolonging drugs that are dose adjusted for renal function.

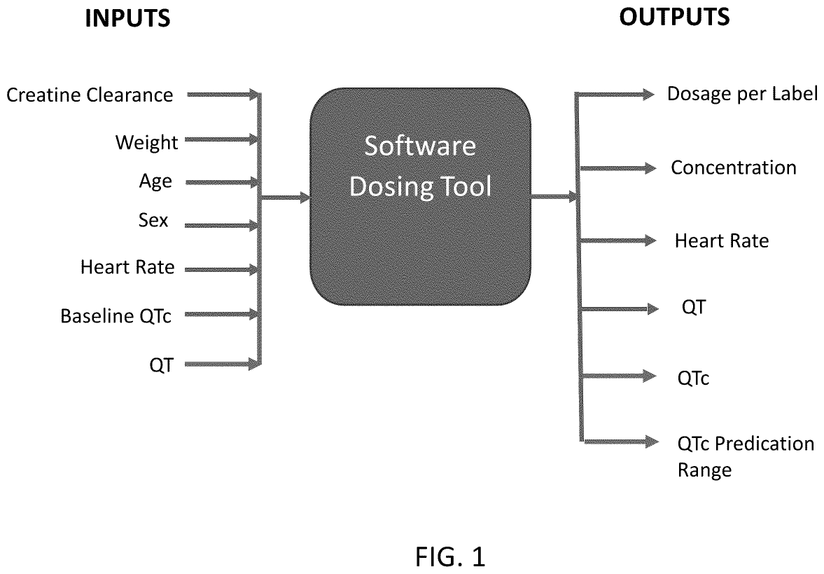

Embodiments include systems and methods of determining an expected QTc for a subject, such as illustrated in FIG. 1. Additional embodiments include dosing methods for prescription drug use, such as dosing methods determined using dosing determination software. Examples include the methods and software described in US2023/0075398, which is incorporated by reference herein in its entirety. Embodiments of the present invention further include methods for predicting QTc for a determined dosing regimen.

Embodiments include methods of performing clinical trial simulations on a PK/PD model for a selected drug to obtain a number of simulated subject profiles, each comprising a simulated QTc range expected to result after administration of the drug to the subject of the simulation. Such simulated profiles can be used to predict the QTc of a subject expected to result from administration of a selected drug.

Methods of the invention are applicable to antiarrhythmic drugs as well as any drug that can have the effect of prolonging QTc and/or that may be affected by a subject's renal function.

Such methods include a method of predicting QTc of a subject, comprising: (a) performing clinical trial simulations on a PK/PD model for a selected drug to obtain a number of simulated subject profiles, each comprising a simulated QTc range expected to result after administration of the drug to the subject of the simulation at a selected amount; (b) obtaining a subject's Baseline QTc and one or more of the subject's Creatinine Clearance, Weight, Age, Sex, and/or Heart Rate before administration of the drug to the subject at the selected amount; (c) optionally determining/selecting a threshold QTc (or allowable % change) for the subject; (d) determining an expected QTc range for the subject based on the simulated subject profiles and the subject's Baseline QTc and one or more of Creatinine Clearance, Weight, Age, Sex, and/or Heart Rate, which expected QTc range would be expected to result after administration of the drug to the subject at the selected amount; (e) optionally visually displaying one or more of the subject's Baseline QTc, a threshold QTc (or allowable % change), an expected median QTc expected to result after administration of the drug to the subject at the selected amount, and/or a 95% confidence interval of the expected QTc range.

In embodiments, a priori profiles are created from data inputs based on simulation results generated from real-world data. The profiles are expected QTc ranges, based on 95% confidence intervals. The profiles can for example be based on initiations, escalations, de-escalations or substitution of IV for oral dosing at 80 mg, 120 mg and 160 mg of sotalol. In embodiments, the profiles can for example be based on initiations, escalations, de-escalations or substitution of IV for oral dosing at 125 μg, 250 μg and 500 μg of dofetilide. In embodiments, the profiles can for example be based on initiations, escalations, de-escalations or substitution of IV for oral dosing at 400 mg/day, 600 mg/day and 800 mg/day of amiodarone. In embodiments, the profiles can for example be based on initiations, escalations, de-escalations or substitution of IV for oral dosing at 250 mg, 500 mg and 1000 mg or procainamide.

In embodiments, clinical simulations can be performed for subjects with normal renal function (CLCR≥90 mL/min), mild renal impairment (CLCR=60-89 mL/min), moderate renal impairment (CLCR=30-59 mL/min), and/or severe renal impairment (CLCR=10-29 mL/min).

In an example, a patient who weighs 112.4 kg, has a creatinine clearance in the range of 30-69 mL/min (such as 35 mL/min), a heart rate of 115 bpm, a baseline QTc of 426 msec, with an a priori profile that suggests that the QTc at/during/after infusion would be expected to rise by 20 to 40 points, if the expected change in QTc (20-40 points) falls above a selected threshold (or above an allowable % change), such as above 500 msec or greater than 20%, this prediction could be used to instruct a physician or other practitioner that administration of the antiarrhythmic drug, e.g., sotalol, in this situation is discouraged. If the change in QTc that is expected is below the selected threshold, then the physician/practitioner would have confidence in proceeding with administering the selected amount of sotalol.

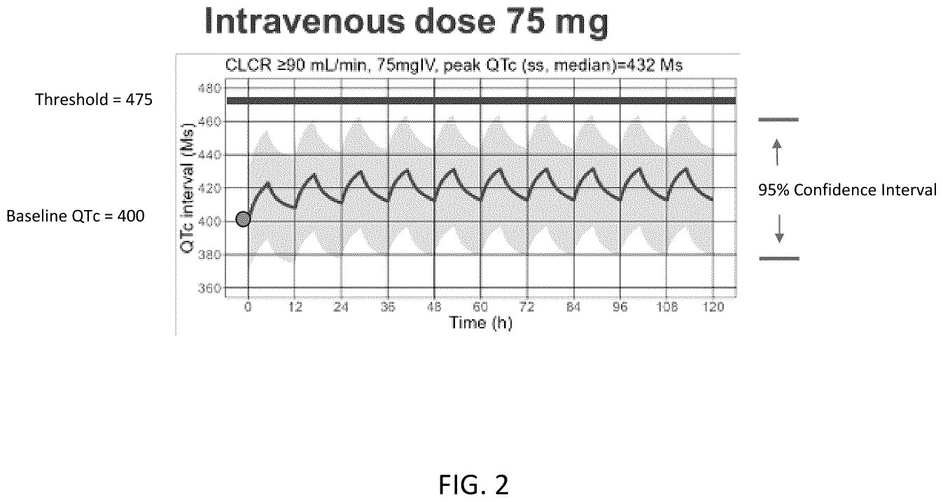

In embodiments, an associated confidence interval, such as a 95% confidence interval, can be graphically displayed for each profile and a patient's baseline QTc can be displayed within this range so that its proximity to an upper threshold could be visually determined. An exemplary graphic display is shown in FIG. 2.

In embodiments, to account for inter-individual variation and uncertainty of the measurement, the Delta Method can be used to obtain an estimator of the variance to account for and/or a Bayesian method could also be used.

As new data is obtained, the profiles can be updated using the same simulation method as was used to create the original simulation and profiles. Machine learning and training of neural networks can also be implemented to improve the accuracy of the prediction methods. In embodiments, the pool of subject profiles can include profiles from actual subjects/patients.

In embodiments, any one or more or all of the following of a patient/subject can be used as inputs: Creatinine Clearance, Weight, Age, Sex, Heart Rate, Baseline QTc, Upper Safety Limit (Threshold and/or allowable % change). In specific embodiments, the inputs include Creatinine Clearance and Baseline QTc. In some embodiments, the inputs include Creatinine Clearance, Weight, Age, Sex and Baseline QTc. Other embodiments include Heart Rate, Baseline QTc and Creatinine Clearance as inputs. In yet other embodiments Weight, Age, Heart Rate and Baseline QTc are inputs. In further embodiments, Baseline QTc is an input.

In embodiments, QTc interval is calculated using the Bazett (equation 1), Fridericia (equation 2), Framingham (equation 3), Hodges (equation 4), or Rautaharju (equation 5) formulas. In the following formulas, “RR” is the interval between two consecutive R waves and “HR” is heart rate.

$$QTc = QT / \sqrt{RR} \qquad ( Equation 1 )$$

$$QTc = QT / {RR}^{1 / 3} \qquad ( Equation 2 )$$

$$QTc = QT + 0.154 \times ( 1 - RR ) \qquad ( Equation 3 )$$

$$QTc = QT + 0.00175 \times ( HR - 60 ) \qquad ( Equation 4 )$$

$$QTc = QT \times ( 120 + HR ) / 180 \qquad ( Equation 5 )$$

In embodiments of the invention, the application programming interface (API) uses an object-oriented approach for model simulation in which each dose is represented by its closed form exponential representation. In embodiments, the actual plasma curve is constructed by superposition (adding the contribution of each individual dose). In embodiments, the speed allows for real-time Monte Carlo simulations. In embodiments, the simulation can be started at any point in time, including at steady state. In embodiments, the time to steady state (which directly follows from the exponential parameters, e.g., half-lives) can be determined. In embodiments, the API is extremely lightweight, making it suitable for online-server applications.

In embodiments, confidence intervals can be established by using the analytical Delta or stochastic Monte Carlo method, or combinations thereof (hybrid). The hybrid simulation method uses the fast Delta method for establishing the points of maximum uncertainty, after which the slower Monte Carlo simulation is executed only at those points. The confidence interval is then indicated using error bars instead of a shaded area.

In embodiments, the software is configured to receive as an input and/or to calculate or determine one or more of: patient type (e.g., adult or pediatric), sex, age, birth date, weight, height, serum creatinine, heart rate, baseline QT and/or QTc, BSA, nCLcr (mL/min/1.73 m²), CLcr (mL/min), type of loading dose (e.g., initiation or escalation), dose patient is currently receiving, current dose frequency, target dose, target dose frequency, medication concentration (such as for IV drug administration), protocol start time (e.g., time of first oral dose or infusion start time for IV dose), timing of one or more additional dose(s), dose amount of one or more additional dose(s), predicted Cmax, and/or predicted QTc.

In embodiments, the software is configured to prompt a user with any warning or disclaimer, such as a black box warning, regarding use of the software and/or drug.

In embodiments, the software is configured to prompt a user when one or more predicted QTc value exceeds a safety threshold (such as contained in a black box warning), such as a threshold of about 500 msec and/or >20% from the baseline QTc value.

In embodiments, the software is configured to prompt (e.g., provide an error message) a user to enter a valid value in response to an attempt to enter an out-of-range value or input.

In embodiments, the software is configured to export a treatment protocol, predicted QTc value(s), and/or treatment summary.

In embodiments, the software is configured to accept one or more dosing inputs and/or one or more QTc prediction inputs; wherein the one or more dosing inputs and/or one or more QTc prediction inputs are chosen from one or more of gender, age, serum creatinine, weight, height, type of loading dose, previous oral dose, previous IV maintenance dose, previous IV loading dose, current oral dose or IV maintenance dose, current dosing interval such as an oral dosing interval, target oral dose, target IV maintenance dose, infusion start time, heart rate, baseline QT interval, and/or baseline QTc interval.

In an embodiment, a user can select “escalation” as the dose type and enter the patient's current oral dose, and the software determines the target oral dose. For example, the user can select “escalation” and enter a current oral dose of 120 mg and the software determines the target oral dose is 160 mg, which is the next higher oral dose for sotalol. Further, for example, the user can select “escalation” and enter a current oral dose of 125 μg and the software determines the target oral dose is 250 μg, which is the next higher oral dose for dofetilide. In further embodiments, the user can select “escalation” and enter a current oral dose of 600 mg/day and the software determines the target oral dose is 800 mg/day, which is the next higher oral dose for amiodarone. For procainamide, for example, the user can select “escalation” and enter a current oral dose of 500 mg and the software determines the target oral dose is 1000 mg, which is the next higher oral dose for procainamide.

### Example 1

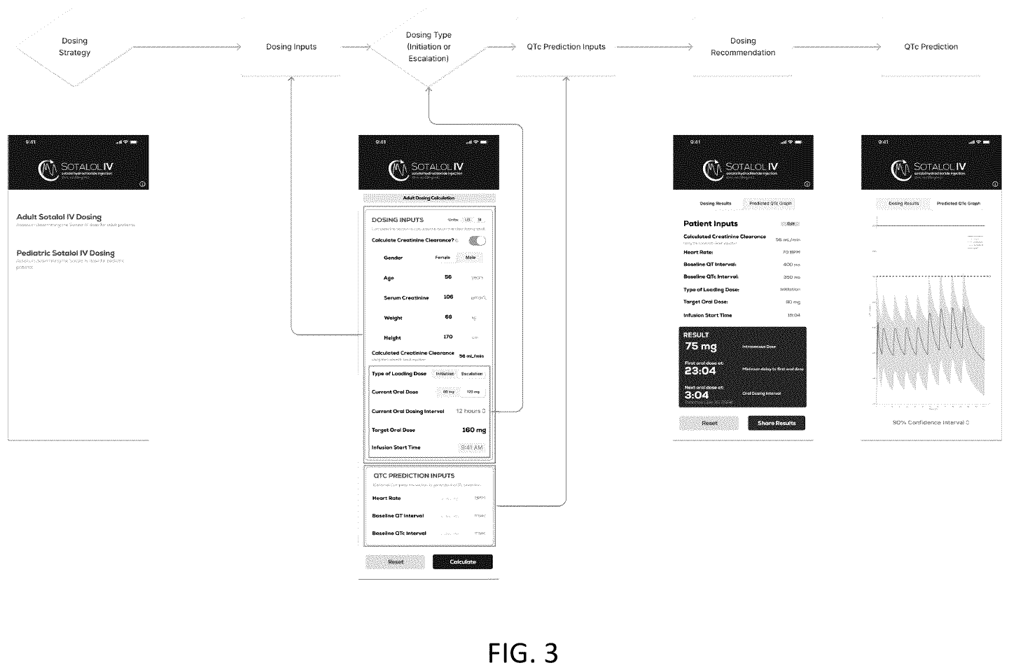

A patient in need of antiarrhythmic drug treatment or therapy, for example sotalol hydrochloride, is admitted for initiation or escalation of the drug while under QTc monitoring. Patient data are inputted into the software (e.g., using a mobile application, such as the mobile application shown in FIG. 3) or medical device for determining predicted QTc or predicted QTc and dosing of the antiarrhythmic drug. Inputs can include one or more of baseline QT (or QTc), weight, age, sex, heart rate, creatinine clearance (or serum creatinine level, which is used by the software to calculate creatinine clearance) of the patient, optionally a start time for the infusion to begin, whether the patient is being initiated or escalated on the drug, and the target oral dose for the patient. For example, for initiating a patient on an antiarrhythmic drug, the target oral or IV dose is input into the software, mobile application, or medical device/system. Product labels can be referenced for various typical target oral doses.

For escalating a patient from one current oral or IV dose to a second higher oral or IV dose, escalation is indicated as well as the target oral dose. For example, if escalation is indicated with a target oral dose of 120 mg of sotalol hydrochloride, then the current oral dose would be understood to be 80 mg (alternatively, or in addition, the app can display the current oral dose whether input by the user or determined by the app). Likewise, if escalation is indicated with a target oral dose of 160 mg, then the current oral dose would be understood to be 120 mg and/or displayed by the app. In embodiments, the software, mobile application, or medical device displays the target oral dose and/or current oral dose.

In embodiments, the patient is optionally connected to a patient monitoring device configured to obtain at least heart rate and/or QT interval and/or QTc data and/or creatinine clearance. In embodiments, the patient monitor is connected with the medical device for determining dosing, such that the heart rate and/or QT interval and/or QTc data, and/or patient creatinine clearance, can be automatically input, or a health professional can input the information into the software, mobile application, or medical device manually from any one or more patient monitoring device or database if desired.

Based on the inputted infusion start time, whether the patient is being escalated or initiated, the patient's creatinine clearance, the target oral dose, and optionally heart rate and/or QT interval and/or QTc data, the medical device or software for determining QTc and/or dosing of the antiarrhythmic drug determines a treatment plan based on stored dosage criteria/protocols. In embodiments, the stored dosage criteria or storage of any patient/subject data or storage of software, the app, or computer-executable instructions can be stored on the medical device or in a different location (than on the medical device) and made available or accessible to the medical device (such as through networking and/or cloud computing). The treatment plan can be presented to and/or approved by a physician remotely or on a system interface, such as from a smartphone or tablet.

In embodiments, the treatment plan additionally comprises predicted QT or QTc values and/or graphs thereof. Using a patient's baseline QTc and one or more of weight, age, sex, heart rate, and/or target IV or oral dose, the software or mobile application derives from a set of rules an expected QTc range for the patient based on simulated profiles and/or actual patient profiles. In embodiments, the treatment management application derives from a set of rules an amount and/or duration of the IV dose to administer based for example on the subject's creatinine clearance and the target oral dose for the subject. The amount and/or duration of the IV dose to administer to the subject can be provided as an output from the treatment management application. Additionally or alternatively, an adjusted amount and/or duration of the IV dose to administer to the subject can be determined and output by the treatment management application. For example, if the treatment management application determines that the amount and/or duration of the IV dose to administer would result in the predicted QT or QTc or QTc range being in an unacceptable range, an adjusted amount and/or duration for the IV dose to administer can be output by the treatment management application as an alternative dosing option for the subject. For example, for a subject being initiated on a 120 mg target oral dose, if the treatment management application determines that the predicted QT or QTc or QTc range would be in an unacceptable range in response to a particular IV dose needed to initiate the subject on the 120 mg oral dose, an alternative dosing protocol can be output where for example the alternative IV dose is a lower amount and the subject is initiated on an 80 mg oral dosing protocol as an alternative option.

The software uses the inputs and stored simulated and/or actual subject profiles, each comprising a QTc or QTc range resulting from or expected to result from administration of sotalol hydrochloride to the actual or simulation subject at a selected amount, to determine an expected QTc or QTc range for the current subject which would result from administration of sotalol hydrochloride to the current subject at the selected amount. In embodiments, predicted QTc is calculated or otherwise determined from the simulated and/or actual subject profiles for a single dose, two doses, three doses, four doses, or five or more doses, whether IV only, oral doses only or a combination of IV doses and oral doses. In embodiments, an expected QTc or QTc range or QTc profile for the subject is not determined by obtaining one or more blood concentration of sotalol hydrochloride for the subject and/or a prior subject.

In embodiments, the software determines the QTc predicted values and confidence interval using an object-oriented approach in which each dose is represented by its closed form exponential representation. The plasma curve is constructed by superposition (i.e., adding the contribution of each individual dose).

In embodiments, real-time Monte Carlo simulations are used. The simulation can be started at any point in time, including at steady state. In other embodiments, the analytical Delta method is used. In still other embodiments, a combination of the stochastic Monte Carlo and analytical Delta methods is used. The results are visually displayed with a shaded confidence interval and/or with error bars. The confidence interval can be set by the user. For example, confidence intervals of 80%, 85%, 90%, or 95% may be selected.

In embodiments, the QTc prediction is presented by the software as a graph with confidence intervals. A QTc safety threshold is set by the user or the software and is optionally shown on the graph. In embodiments, the threshold is 500 msec or 20% from the baseline QTc. For the situation where none of the predicted QTc values reach the safety threshold, the software instructs the treatment can proceed.

A first IV loading dose of antiarrhythmic drug is administered to the patient based on the treatment plan determined by the medical device or software for determining the dosing of the antiarrhythmic drug. The administering can be performed/controlled directly or indirectly by an administration system, for example, by controlling an infusion pump to deliver the antiarrhythmic drug to the patient. The device/system can also be configured to provide instructions to a health professional regarding the treatment plan such that the health professional can control the infusion pump to administer the antiarrhythmic drug to the patient according to the treatment plan. Included in the outputs of the treatment plan are the amount of the IV dose, the time of the first oral dose and the oral dosing interval or time or subsequent oral dose(s), if any. The dose amount and administration time are provided by the treatment plan and are based on creatine clearance or liver clearance capability. In the case of sotalol hydrochloride, the treatment plan is based on the patient's creatinine clearance or creatinine level, whether the patient is being initiated or escalated on the drug, the target oral or IV maintenance dose, and the projected start time for the protocol to begin, i.e., the start time for IV infusion. In embodiments, the treatment plan includes one or more predicted QTc value(s) for one or more doses of the treatment plan. The time for oral dosing to begin is based on the patient's creatinine clearance and the start time of the infusion. The oral dosing interval is based on the patient's creatinine clearance or creatinine level, and a start time for oral doses subsequent to the first oral dose is based on the start time of the first oral dose. If a different start time than the initially projected start time for infusion is needed, then the dosing protocol can be recalculated based on a new input of an actual start time for infusion and/or a new projected start time for infusion. If more than one IV dosing option is appropriate, then a choice of IV dosing options can be presented/outputted.

In embodiments, the patient monitor can obtain heart rate and/or QT intervals either continuously or at time periods established by the treatment plan according to the dosing protocol or the heart rate and/or QT intervals or QTc can be input directly into the software. Based on the QT interval response to the antiarrhythmic drug being administered, additional IV doses of antiarrhythmic drug are administered and/or oral doses of antiarrhythmic drug are administered/instructed by the system. Based on the QT interval response, the IV and/or oral dosages are adjusted accordingly, for example, the dosage amount can be decreased, increased, or halted. In embodiments, the administration system is configured to halt treatment, or provide instructions for halting treatment, and notify a system user, for example by way of an alarm or alert such as a text alert, if the patient's heart rate or QT interval deviate from predetermined accepted values. The administration system and/or software is additionally configured to update the treatment plan and/or QTc prediction based on the QT interval response during and after administration of each dose of antiarrhythmic drug. If the treatment plan and/or QTc plan is updated by the system and/or software, the system and/or software can prompt a user for approval prior to further administration of antiarrhythmic drug.

### Example 2

In embodiments of the invention, the software, mobile application, and/or treatment system is capable of receiving various inputs and calculating/determining various values. In embodiments, not all inputs are required for dose determination and/or QTc Prediction.

TABLE 1
Example Inputs/Outputs for Dose Determination and QTc Prediction

| Input/Output | Values | Units | Entered/Determined |
|---|---|---|---|
| Patient Type | Pediatric/Adult | — | Entered/Determined |
| Sex | Male/Female | — | Entered |
| Age | 0-120 | Days/ | Entered |
| | | months/ | |
| | | years | |
| BSA | 0-20 | m² | Entered/Determined |
| Serum Creatinine | 0-135 | μmol/L | Entered |
| | | or mg/dL | |
| Creatinine Clear. | 0-150 | mL/min | Entered/Determined |
| (CLcr) | | | |
| nCLcr | 0-150 | mL/min/ | Entered/Determined |
| | | 1.73 m² | |
| Weight | 0-500 | kg | Entered |
| Height | 0-250 | cm | Entered |
| Type of Initial Dose | Initiation, | — | Entered/Determined |
| | Escalation, or | | |
| | Substitute | | |
| | for Oral | | |
| Administration | NPO/PO | — | Entered |
| Restrictions | | | |
| Current Dose | 0/80/120/160 | mg | Entered/Determined |
| Target Oral Dose | 80/120/160/240 | mg | Entered/determined |
| Loading Dose Amount | 0-240 | mg | Determined |
| Infusion/Dosing Start | 0:00-23:59 | — | Entered |
| Time | | | |
| Heart Rate | 40-220 | BPM | Entered/Determined |
| Baseline QT Interval | 350-500 | msec | Entered/Determined |
| Baseline QTc Interval | 350-500 | msec | Entered/Determined |
| Subsequent Dose Type | IV/Oral | — | Entered |
| Subsequent Dose Time | 0:00-23:59 | — | Entered/Determined |
| Dosing Interval | 0-48 | hours | Entered/Determined |

In embodiments of the invention, a user enters one or more values for one or more inputs listed in Table 1. In some embodiments, one or more of the inputs are calculated/determined by the software/system based on other inputs entered by the user.

In embodiments, patient type is entered by the user or is determined based on a user entered patient age and/or date of birth. In embodiments, patient age is entered by the user or is determined by the user entered date of birth.

In embodiments, body surface area (BSA) is entered by the user or is determined based on the entered patient height and weight.

In embodiments, creatinine clearance (CLcr) and/or normalized creatinine clearance (nCLcr) are calculated based on an entered serum creatinine and based on one or more of patient age, sex, height, weight, or combinations thereof. In other embodiments, CLcr and/or nCLcr is known and entered by the user.

In embodiments, the type of initial dose is entered by the user or is determined based on an entered administration restriction, such as NPO status.

In embodiments, the loading dose amount is determined based on current dose, target dose, baseline QT or QTc interval, age, CLcr, or combinations thereof.

In embodiments, one or more of the heart rate, QT interval, and/or QTc interval are entered by a user or determined by the system based on analysis of a patient ECG. In embodiments, QTc is determined by the system based on a heart rate and QT interval entered by a user.

In embodiments, the subsequent dose time is determined based on one or more of: infusion/dosing start time, CLcr, age, target dose, QT or QTc interval, and/or type of initial dose.

In embodiments, the dosing interval is determined based on one or more of: CLcr, age, target dose, and/or QT or QTc interval.

In embodiments, if a user attempts to enter an out-of-range value or has entered incomplete information (e.g., missing criteria critical to calculation), the software will provide the user with an error message to guide them to correct the error.

### Example 3

In an example treatment, a female patient, age 50, is to be initiated on sotalol treatment. The patient is diagnosed with atrial fibrillation and is currently in normal sinus rhythm. Upon admission to the hospital, a serum creatinine level and baseline QT interval are measured and entered into the software/system. Additionally or alternatively, any one or more of the patient data listed in Table 2 can be provided as inputs from which to base one or more outputs of the software/system on.

A target oral dose amount of 80 mg is decided by a physician and entered into the system/software. Based on one or more of the patient data inputs, the system/software is configured to determine a treatment protocol and a predicted QTc interval for one or more doses of the treatment protocol.

In embodiments, the treatment protocol includes a loading dose amount, dosing interval, and optionally a subsequent dose time determined from one or more of: infusion start time, target oral dose, creatinine clearance, and/or baseline QTc.

TABLE 2
Input/Output options according to an
example embodiment of the invention

| Input/Output | Values | Units |
|---|---|---|
| Patient Type | Adult | — |
| Sex | Female | — |
| Age | 50 | years |
| BSA | 1.81 | m² |
| Serum Creatinine | 1.8 | mg/dL |
| Creatinine Clear. (CLcr) | 41 | mL/min |
| nCLcr | 43 | mL/min/1.73 m² |
| Weight | 70 | kg |
| Height | 170 | cm |
| Type of Initial Dose | Initiation | — |
| Administration | PO | — |
| Restrictions | | |
| Current Dose | 0 | mg |
| Target Oral Dose | 80 | mg |
| Loading Dose Amount | 75 | mg |
| Infusion Start Time | 8:00 | — |
| Heart Rate | 75 | BPM |
| Baseline QT Interval | 375 | msec |
| Baseline QTc Interval | 404 | msec |
| Subsequent Dose Type | Oral | — |
| Subsequent Dose Time | 14:00 | — |
| Dosing Interval | 24 | hours |

### Example 4

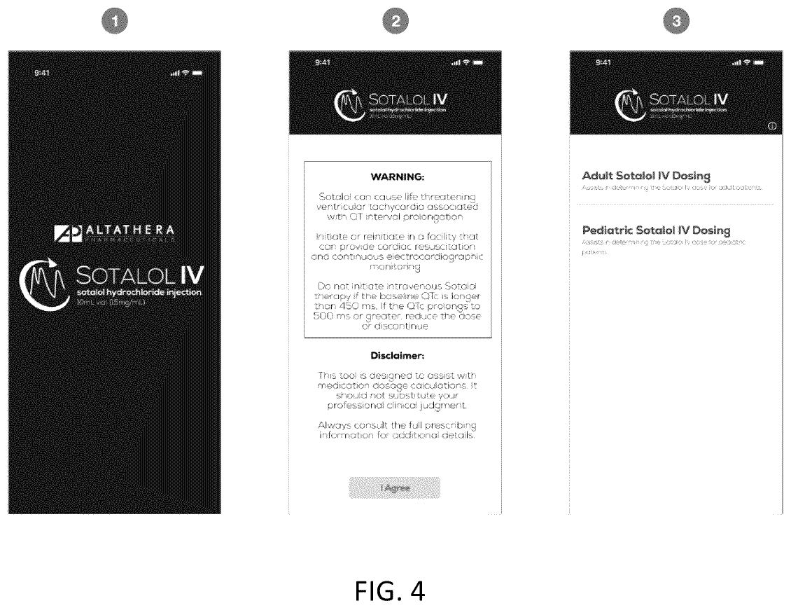

As shown in FIG. 4, the user, upon entering the mobile application or software, will select the adult or pediatric dosing strategy. The user will enter one or more patient-related inputs (or dosing inputs), including but not limited to one or more of gender, age, date of birth, serum creatinine, weight, height, body surface area, creatinine clearance, baseline QTc, type of loading dose (such as initiation or escalation), current oral dose (if any), current oral dosing interval (if any), target oral dose and/or an infusion start time. An option can be provided for the user to indicate whether it is desired for the mobile application to calculate a creatinine clearance from various patient-related inputs, otherwise a measured creatinine clearance of the patient/subject can be provided/entered by the user. An option can be provided for the patient-related inputs to be of a specific units, such as US or SI. An information icon can be made available to the user at various user interface screens or at anytime during use of the mobile application for providing prescribing information to the user, such as a drug product label.

In embodiments, if creatinine clearance is not entered by the user, it will be calculated by the mobile application or software.

If QTc prediction is desired, the user will enter additional inputs for QTc prediction, such as heart rate, baseline QT interval, and/or baseline QTc interval.

The software or mobile application will display the recommended IV dose and the oral dose administration times for the target oral dose, such as the times for administering a first oral dose and one or more subsequent oral dose and/or a dosing interval.

Where QTc prediction is desired, the software or mobile application will display a graph showing the predicted QTc profile for the patient based on the recommended IV dose and/or one or more of the oral doses. In embodiments, a predicted heart rate can also be presented to the user. In embodiments, the predicted QTc profile comprises QTc values predicted for multiple doses. In embodiments, the predicted QTc profile includes a confidence interval, which is optionally selected by the user (e.g., 90%, 95%, etc.). In embodiments, the predicted QTc graph provides a maximum QTc threshold (which can be entered by the user and/or a default value) and optionally displays a warning if any of the predicted QTc values exceed the threshold.

### Example 5

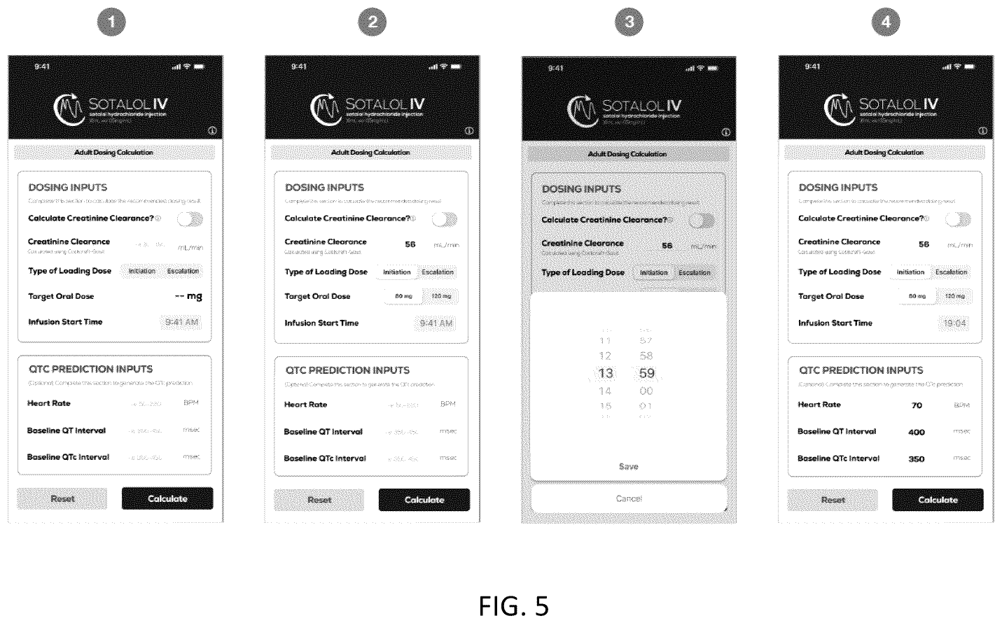

The user, upon entering the mobile application, selects the adult dosing strategy. The user enters a creatinine clearance of 56 mL/min, selects the “initiation” loading dose type, selects a target oral dose of 80 mg, and enters a planned infusion start time of 19:04 (FIG. 5).

The user enters QTc prediction inputs including a heart rate of 70 bpm, a baseline QT interval of 400 msec, and baseline QTc interval of 350 msec.

QTc prediction and dosing strategy determination are performed by the software (see, for example, Example 8) and presented to the user.

### Example 6

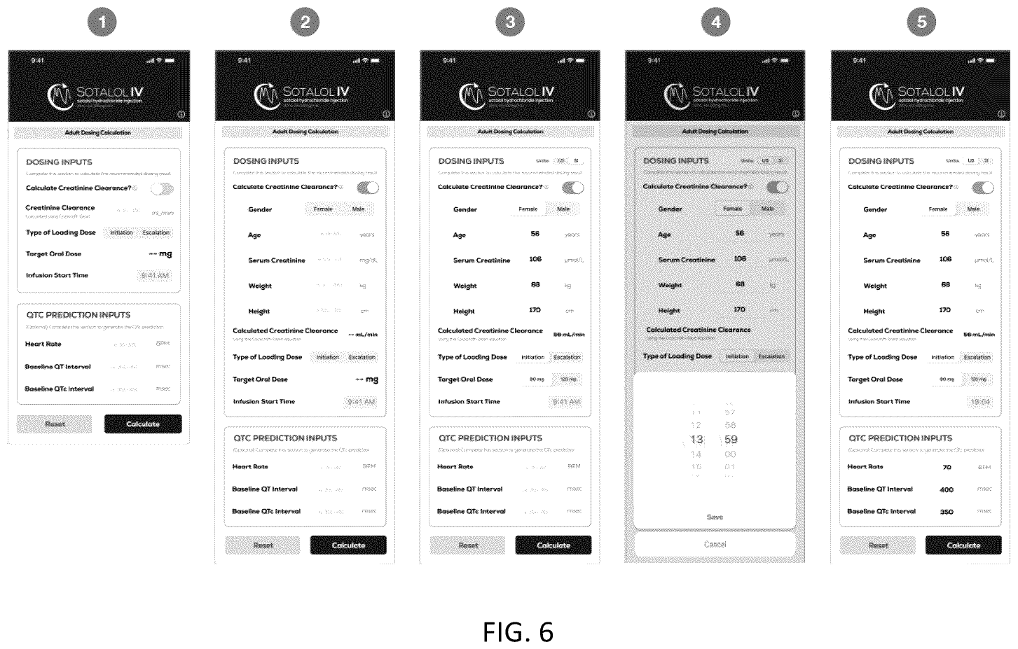

The user, upon entering the mobile application, selects the adult dosing strategy. The user selects the “calculate creatinine clearance” option. This selection prompts the user to enter gender, age, serum creatinine, weight, and/or height. The user indicates the patient is female, age 56, 68 kg, 170 cm tall, and has a serum creatinine of 106 μmol/L. The software uses this information to calculate a creatinine clearance of 56 mL/min (FIG. 6).

The user selects initiation as the type of loading dose and 80 mg for the target oral dose. The user enters an infusion start time of 19:04.

The user further enters QTc prediction inputs, including a heart rate of 70 bpm and a baseline QT interval of 400 msec. From one or more or all of the inputs, the software determines the baseline QTc interval is 350 msec.

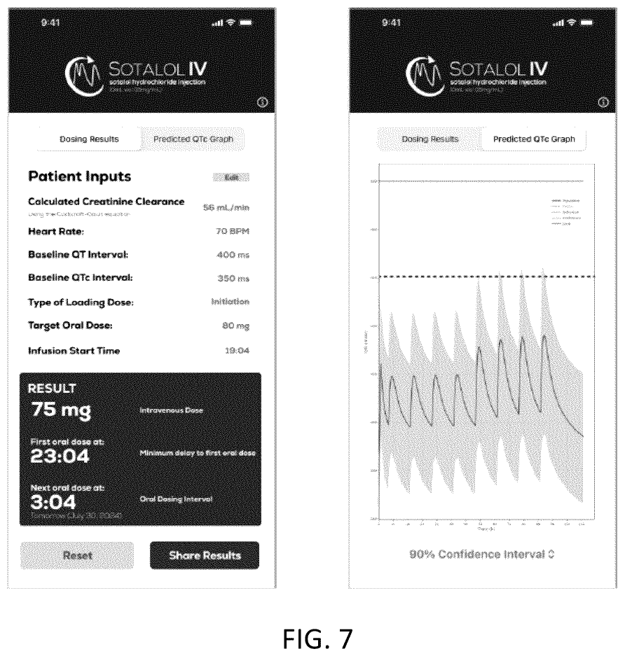

A dosing strategy determination (see, for example, Example 1) and QTc prediction (see, for example, Example 8) for the dosing strategy are performed by the software and presented to the user (FIG. 7).

### Example 7

The user selects the pediatric dosing strategy in the treatment management application. The user enters the patient's date of birth and the software determines the patient's age is 8 days and/or the user can enter the patient's age. The user toggles the creatinine clearance calculation switch to display the creatinine clearance inputs. The user selects “male” for patient gender and enters a serum creatinine of 0.63 mg/dL and a height of 63 cm. Based on the inputs, the software determines the creatinine clearance for the patient is 41 mL/min/1.73 m². Alternatively, the creatinine clearance for the patient is entered by the user.

Based on the pediatric patient's age and creatinine clearance, a dosing strategy is determined by the treatment management application. In embodiments, the treatment management application comprises or is in operable communication with a look-up table for determining the amount of IV sotalol appropriate for the pediatric patient based on the patient's age and creatinine clearance. In embodiments, the dosing amounts from Table 3 can be used.

TABLE 3
Dosing (mg/kg) in Children According
to Renal Function and Age

| | CrCl | |
|---|---|---|
| | (mL/min/ | Dose |
| Age | 1.73 m²) | (mg/kg) |
| 0.1 month | ≥33 | 0.1-0.5 |
| | 22-32 | 0.05-0.45 |
| | 11-22 | 0.05-0.35 |
| | 4-11 | 0.01-0.25 |
| >0.1 to | ≥36 | 0.7-1.2 |
| 0.5 month | 24-35 | 0.5-1 |
| | 12-24 | 0.25-0.75 |
| | 4-12 | 0.1-0.6 |
| >0.5 to | ≥40 | 0.95-1.5 |
| 1 month | 26-39 | 0.7-1.25 |
| | 13-26 | 0.45-0.95 |
| | 4-13 | 0.2-0.7 |
| >1 to 3 | ≥53 | 1-2 |
| months | 35-53 | 0.75-1.75 |
| | 18-35 | 0.3-1.3 |
| | 6-17 | 0.1-1.1 |
| >3 to | ≥68 | 0.9-1.9 |
| 6 months | 56-67 | 0.6-1.6 |
| | 23-44 | 0.3-1.3 |
| | 8-22 | 0.1-1.1 |
| >6 to | ≥81 | 0.9-1.9 |
| 12 months | 54-80 | 0.6-1.6 |
| | 27-53 | 0.3-1.3 |
| | 9-26 | 0.1-1.1 |
| >1 to | ≥90 | 0.75-1.75 |
| 2 years | 60-89 | 0.5-1.5 |
| | 30-59 | 0.2-1.2 |
| | 10-29 | 0.1-1 |
| >2 to | ≥90 | 0.7-1.7 |
| 6 years | 60-89 | 0.4-1.5 |
| | 30-59 | 0.15-1.15 |
| | 10-29 | 0.05-0.95 |
| >6 to | ≥90 | 0.4-1.4 |
| 12 years | 60-89 | 0.2-1.2 |
| | 30-59 | 0.1-1.1 |
| | 10-29 | 0.05-0.9 |

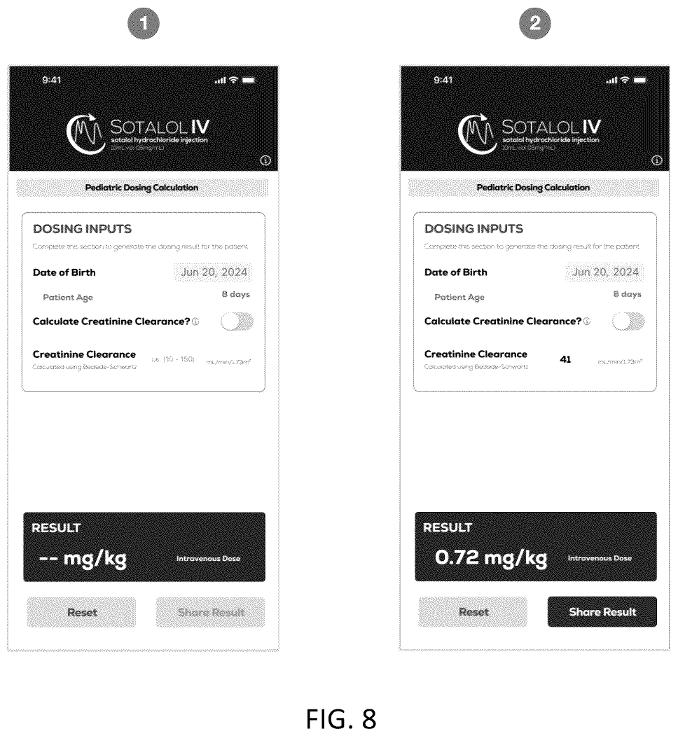

From the patient-related data (age=8 days, and CrCl=41 mL/min/1.73 m²) and by referring to Table 3, the software determines the starting IV dose is 0.72 mg/kg (FIG. 8). The determined dosing amount can be derived by the software/system from one or more patient-related inputs and/or a look up table, such as Table 3, or can be derived by the software/system and confirmed by being within an acceptable range provided by a look up table, such as Table 3. In embodiments, the user can enter the patient weight and the starting IV dose can be displayed in milligrams. In embodiments, QTc prediction can be performed by the software and presented to the user derived from the dosing strategy determined for the patient.

### Example 8

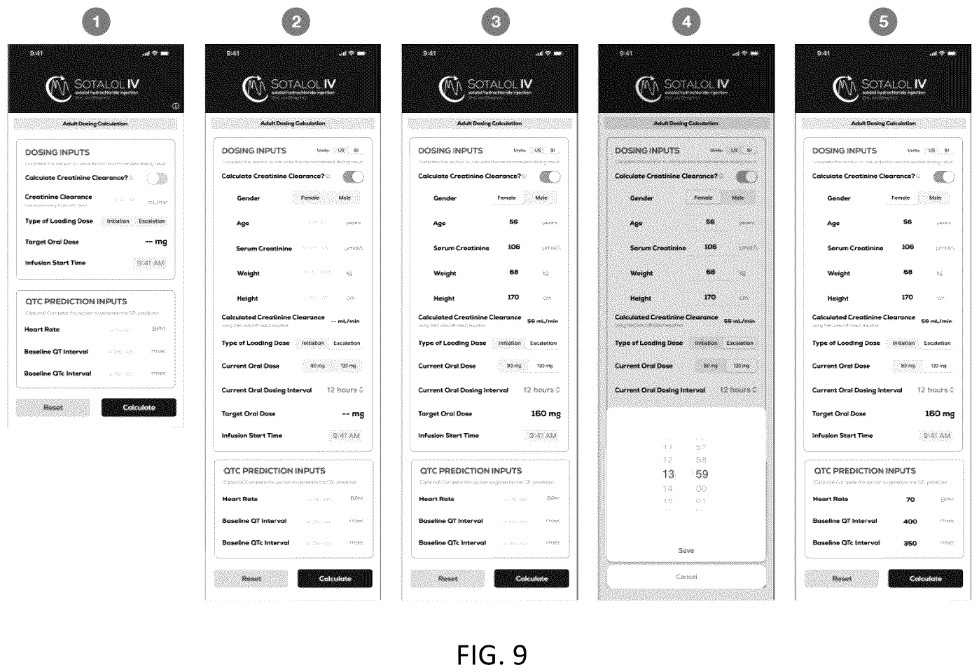

The software or mobile application is used to prepare a QTc prediction for a patient to be escalated (by way of administration of a 1 hour IV dose of sotalol) from oral dosing of 120 mg B.I.D. sotalol hydrochloride to 160 mg B.I.D. oral dosing (FIG. 9). In any embodiment herein, QTc prediction can be used alone or in combination with dosing determination and vice versa.

The user inputs one or more of patient age, weight, sex, heart rate, creatinine clearance, and/or baseline QTc into the app and/or based on user inputs one or more patient-related characteristic (such as creatinine clearance) is calculated or otherwise determined. In embodiments, a user can enter the amount of one or more IV dose and/or one or more oral dose of a drug (such as sotalol) to be administered to the patient.

In embodiments, the software uses the inputs to determine the appropriate amount of IV sotalol to administer to achieve the desired escalation from the 120 mg oral dose to the target 160 mg oral dose. For example, in embodiments, the appropriate amount of IV sotalol to administer can be selected to expose the patient to a QTc expected from multiple sequential oral doses of 160 mg sotalol hydrochloride.

The stored simulated and/or actual subject profiles that comprise a QTc or QTc range resulting from or expected to result from administration of sotalol hydrochloride to the subject of the profile using the same IV loading dose amount are used to determine an expected QTc or QTc range for the current subject which would result from administration of the same IV loading dose amount of sotalol hydrochloride to the current patient.

The software determines the QTc predicted values and optionally a confidence interval using an object-oriented approach in which each dose is represented by its closed form exponential representation. The plasma curve is constructed by superposition (i.e., adding the contribution of each individual dose).

In embodiments, real-time Monte Carlo simulations are used. The simulation can be started at any point in time, including at steady state.

In other embodiments, the analytical Delta method is used. In still other embodiments, a combination of the stochastic Monte Carlo and analytical Delta methods is used.

The results are visually displayed with a shaded confidence interval and/or with error bars. The confidence interval can be set by the user. For example, confidence intervals of 80%, 85%, 90%, or 95% may be selected.

If the predicted QTc exceeds a pre-set safety threshold (e.g., ≥20% from baseline QTc), the software recommends against administration. If the predicted QTc falls within the safety threshold, the software can prompt the user with any additional inputs needed to prepare a dosing protocol and/or generate a dosing protocol upon user request.

### Example 9

### Antiarrhythmic Drug Administration System

A patient in need of antiarrhythmic drug treatment or therapy, for example sotalol hydrochloride treatment, is admitted for initiation or escalation of the drug while under QTc monitoring. Patient-related data are inputted into the medical device for determining dosing of the antiarrhythmic drug and/or QTc prediction based on the dosing. Patient-related data includes one or more of a creatinine clearance or creatinine level of the patient, optionally a start time for the infusion to begin, whether the patient is being initiated or escalated on the drug, and the target oral dose or maintenance IV dose for the patient.

For example, for initiating a patient on an antiarrhythmic drug, the target oral or IV maintenance dose is input into the medical device and/or the desired IV loading dose can alternatively or in addition be entered into the medical device. For escalating a patient from one current oral or IV maintenance dose to a second higher oral or IV maintenance dose, i) escalation is indicated as well as the target oral dose or the target IV maintenance dose, or ii) escalation and the current oral dose or the current IV maintenance dose are indicated, iii) or the current oral or IV maintenance dose are indicated in combination with the target oral or target IV maintenance doses. For example, if escalation is indicated with a target oral dose of 120 mg of sotalol hydrochloride, then the current oral dose would be understood to be 80 mg. Likewise, if escalation is indicated with a target oral dose of 160 mg, then the current oral dose would be understood to be 120 mg. If a current oral dose of 80 mg is indicated and a target oral dose of 120 mg is indicated, then it would be understood that an escalation protocol is desired. In embodiments, the current oral or IV maintenance dose, the target oral or IV maintenance dose, and escalation can be indicated.

In embodiments, patient-related data is used by the medical device to determine a predicted QTc profile that would result from the determined treatment. For example, a baseline QTc for the patient can be entered into the medical device. The predicted QTc profile is optionally displayed as a graph with confidence intervals. The medical device optionally compares the predicted QTc profile with a QTc safety threshold. If any predicted QTc value exceeds the safety threshold, the medical device is configured to display a warning recommending the treatment is not recommended. If the predicted QTc profile is acceptable, dosing can proceed.

In embodiments, the patient is optionally connected to a patient monitoring device configured to obtain at least heart rate and/or QT interval and/or QTc data and/or creatinine clearance. In embodiments, the patient monitor is connected with the medical device for determining dosing and/or predicted QTc, such that the heart rate and/or QT interval and/or QTc data, and/or patient creatinine clearance, can be automatically input, or a health professional can input the information into the medical device manually from any one or more patient monitoring device or database if desired.

Based on whether the patient is being escalated or initiated, the patient's creatinine clearance, the target oral dose, and optionally heart rate and/or baseline QT interval and/or baseline QTc data, the medical device for determining dosing of the antiarrhythmic drug determines a treatment plan based on stored dosage criteria/protocols and/or a predicted QTc based on the treatment plan. In embodiments, the stored dosage criteria or storage of any patient/subject data or storage of software, the app, or computer-executable instructions can be stored in a different location (than on the medical device) and made available or accessible to the medical device (such as through networking and/or cloud computing). The treatment plan can be approved by a physician remotely or on a system interface, such as from a smartphone or tablet.

A first IV loading dose of antiarrhythmic drug is administered to the patient based on the treatment plan determined by the medical device for determining the dosing of the antiarrhythmic drug. The administering can be performed/controlled directly or indirectly by an administration system, for example, by controlling an infusion pump to deliver the antiarrhythmic drug to the patient. The device/system can also be configured to provide instructions to a health professional regarding the treatment plan such that the health professional can control the infusion pump to administer the antiarrhythmic drug to the patient according to the treatment plan. Included in the outputs of the treatment plan are the amount of the IV dose (such as a loading dose), the time of the first oral dose (or time after administration of the IV dose) and the oral dosing interval of or time of subsequent oral dose(s) if any. The dose amount(s) and administration time(s)/timing are provided by the treatment plan and are based on creatine clearance or liver clearance capability. In the case of sotalol hydrochloride, the treatment plan is based on the patient's creatinine clearance or creatinine level, whether the patient is being initiated or escalated on the drug, the target oral dose, and/or the projected start time for the protocol to begin, i.e., the start time for IV infusion. The time for oral dosing to begin is based on the patient's creatinine clearance and the start time or finish time of the infusion. The oral dosing interval (e.g., timing between oral doses) is based on the patient's creatinine clearance or creatinine level, and a start time for oral doses subsequent to the first oral dose is based on the start time of the first oral dose. If a different start time than the initially projected start time for infusion is needed, then the dosing protocol can be recalculated based on a new input of an actual start time for infusion and/or a new projected start time for infusion. If more than one IV dosing option is appropriate, then a choice of IV dosing options can be presented/outputted.

In embodiments, the patient monitor can obtain heart rate and/or QT intervals either continuously or at time periods established by the treatment plan according to the dosing protocol. Based on the QT interval response to the antiarrhythmic drug being administered, additional IV doses of antiarrhythmic drug are administered and/or oral doses of antiarrhythmic drug are administered/instructed by the system. Based on the QT interval response, the IV and/or oral dosages are adjusted accordingly, for example, the dosage amount can be decreased, increased or halted. In embodiments, the administration system is configured to halt treatment, or provide instructions for halting treatment, and notify a system user, for example by way of an alarm or alert such as a text alert, if the patient's heart rate or QT interval deviate from predetermined accepted values. The administration system is additionally configured to update the treatment plan based on the QT interval response during and after administration of each dose of antiarrhythmic drug. If the treatment plan is updated by the system, the system can prompt a user for approval prior to further administration of antiarrhythmic drug.

### Example 10

### Example Sotalol Protocol:

Dosing criteria for the device and/or system of any example above can include criteria for infusing a loading dose of sotalol for a period of 1-5 hours, as well as criteria for monitoring a patient's QTc at 15-minute intervals during the infusion. If the baseline QTc is >450 ms (JT>330 ms if QRS over 100 ms), sotalol is not recommended.

Monitoring of QTc is continued around Tₘₐₓ (2 to 4 hours post-dose) following the first oral dose (in all patients) and second oral dose (in patients with CrCl≥60 mL/min).

If the QTc prolongs to ≥500 ms or increases 20% or more from baseline (or would be expected to prolong) when initiating for an oral dose of 80 mg, the dosing criteria involves discontinuing the drug; if initiating for an oral dose of 120 mg the dosing criteria involves discontinuing the drug and considering a lower dose, such as continuing with administering the patient's prior 80 mg oral dosing protocol. If re-initiation at a lower dose of 80 mg is desired, the dosing criteria involves waiting at least 1 day (in patients with CrCl≥60 mL/min), or at least 3 days (in patients with CrCl≥30 to <60 mL/min), or 7 days (in patients with CrCl≥10 to <30 mL/min). Similarly, when escalating from 120 mg to a 160 mg oral dosing protocol, if the QTc prolongs to ≥500 ms or increases 20% or more from baseline (or would be expected to prolong), administration of the IV can be halted and the patient can resume taking the 120 mg doses.

The dosing criteria of the administration system comprises amounts of various intravenous loading doses, the dosing interval(s) for oral or IV maintenance administration, and the minimum delay (if any) between the end of the infusion and the first oral or IV maintenance dose, which depend on the target oral or IV dose and creatinine clearance; see Table 3.

TABLE 4
Recommended Loading Dose

| | | | | | Minimum | |
|---|---|---|---|---|---|---|
| | Intravenous loading dose [mg] to be administered over 1 hour | | | | delay | Oral |
| Creatinine | when the oral dose is going from . . . | | | | to first | dosing |
| Clearance* | Sotalol Initiation | | Sotalol Escalation | | oral dose | interval |
| [mL/min] | 0 to 80 mg** | 0 to 120 mg | 80 to 120 mg | 120 to 160 mg | [hours] | [hours] |
| >90 | 60 | 90 | 75 | 90 | 4 | 12 |
| 60-90 | 82.5 | 125 | 82.5 | 105 | 4 | 12 |
| 30-60 | 75 | 112.5 | 82.5 | 105 | 6 | 24 |
| 10-30 | 75 | 112.5 | 82.5 | 105 | 12 | 48 |

*Calculated using Cockcroft Gault formula
**Recommended starting dose

### Example 11

### Sotalol Administration System

A patient is identified for whom initiation or escalation of sotalol is desired while under QTc monitoring. For initiating a patient on sotalol, the target oral sotalol dose is input into the administration system. For escalating a patient from a current/previous oral sotalol dose (e.g., 80 mg or 120 mg) to a second higher oral sotalol dose (e.g., respectively 120 mg or 160 mg), escalation is input into the administration system along with the target oral sotalol dose and/or the current oral dose. Based on the inputted patient data, the medical device for determining dosing of the antiarrhythmic drug determines a treatment plan based on dosage criteria/protocols. In embodiments, the treatment plan includes QTc prediction for one or more doses of sotalol.

The dosage criteria/protocols comprise the following rules relating to determining the appropriate IV loading dose for a particular patient:

Dosing based on administering a loading dose of sotalol hydrochloride over a period of 1 hour, wherein the IV loading dosage criteria is selected from dosages I(a)-IV(d):
I for patients having a creatinine clearance (CrCl) of >90 mL/min;
(a) 60 mg for a patient naïve to sotalol and having a target of 80 mg;
(b) 90 mg for a patient naïve to sotalol and having a target of 120 mg;
(c) 75 mg for a patient previously receiving 80 mg of sotalol;
(d) 90 mg for a patient previously receiving 120 mg of sotalol;
II for patients having a creatinine clearance (CrCl) of 60 to 90 mL/min;
(a) 82.5 mg for a patient naïve to sotalol and having a target of 80 mg;
(b) 125 mg for a patient naïve to sotalol and having a target of 120 mg;
(c) 82.5 mg for a patient previously receiving 80 mg of sotalol;
(d) 105 mg for a patient previously receiving 120 mg of sotalol;
III for patients having a creatinine clearance (CrCl) of 30 to 60 mL/min;
(a) 75 mg for a patient naïve to sotalol and having a target of 80 mg;
(b) 112.5 mg for a patient naïve to sotalol and having a target of 120 mg;
(c) 82.5 mg for a patient previously receiving 80 mg of sotalol;
(d) 105 mg for a patient previously receiving 120 mg of sotalol;
IV for patients having a creatinine clearance (CrCl) of 10 to 30 mL/min;
(a) 75 mg for a patient naïve to sotalol and having a target of 80 mg;
(b) 112.5 mg for a patient naïve to sotalol and having a target of 120 mg;
(c) 82.5 mg for a patient previously receiving 80 mg of sotalol;
(d) 105 mg for a patient previously receiving 120 mg of sotalol.

In embodiments, based on a particular dosing protocol identified for a patient, a corresponding predicted QTc can be determined for the patient. If the predicted QTc is within an acceptable range, administration of sotalol can begin. A first IV loading dose of sotalol is administered to the patient based on the above dosing criteria applicable to the patient. For example, the IV loading dose according to protocol I(a) would be determined by the device or system as an appropriate loading dose for a patient who is naïve to sotalol, with a target oral dose of 80 mg and having a creatinine clearance in the range of >90 mL/min. The administering can be performed/controlled directly or indirectly by any component of the administration system, for example, by controlling an infusion pump to deliver the antiarrhythmic drug to the patient. The system can also be configured to provide instructions to a health professional regarding the treatment plan such that the health professional can control the infusion pump to administer the antiarrhythmic drug to the patient according to the treatment plan.

In embodiments, optionally the patient monitor continues to obtain heart rate and/or QT intervals either continuously or at time periods established by the treatment plan according to the dosing protocol. If the QTc prolongs to >500 ms or increases 20% from baseline when initiating for an oral dose of 80 mg, the dosing criteria involves discontinuing the drug; if initiating for an oral dose of 120 mg the dosing criteria involves discontinuing the drug and considering a lower dose. If re-initiation at a lower dose of 80 mg is desired, the dosing criteria involves waiting at least 1 day (in patients with CrCl≥60 mL/min), or at least 3 days (in patients with CrCl≥30 to <60 mL/min), or 7 days (in patients with CrCl≥10 to <30 mL/min).

Based on the QT interval response to the antiarrhythmic drug being administered, oral doses of antiarrhythmic drug are administered/instructed by the system according to dosing criteria adhering to the following rules:

Dosing based on orally administering sotalol hydrochloride to the patient, wherein the dosing criteria of the administration system are selected from the following dosage/intervals selected from (I)-(IX):
I. 80 mg BID, starting 4 hours after completion of IV doses I(a) or II(a);
II. 120 mg BID, starting 4 hours after completion of IV doses I(b), I(c), II(b), or II(c);
III. 160 mg BID, starting 4 hours after completion of IV doses I(d) or II(d);
IV. 80 mg QD, starting 6 hours after completion of IV dose III(a);
V. 120 mg QD, starting 6 hours after completion of IV doses III(b) or III(c);
VI. 160 mg QD, starting 6 hours after completion of IV dose III(d);
VII. 80 mg at a 48 h interval, starting 12 hours after completion of IV dose IV(a);
VIII. 120 mg at a 48 h interval, starting 12 hours after completion of IV doses IV(b) or IV (c);
IX. 160 mg at a 48 h interval, starting 12 hours after completion of IV dose IV(d).

Based on the QT interval response, the medical device or system can indicate that IV and/or oral dosages should be adjusted accordingly, for example, the dosage amount can be decreased, increased or halted. In embodiments, the administration system is configured to halt treatment, or provide instructions for halting treatment, and notify a system user, for example by way of an alarm or alert such as a text alert, if the patient's heart rate or QT interval deviate from predetermined accepted values. The administration system can also be configured to update the treatment plan based on the QT interval response during and/or after administration of each dose of antiarrhythmic drug.

The present disclosure has described particular implementations having various features. In light of the disclosure provided herein, it will be apparent to those skilled in the art that various modifications and variations can be made without departing from the scope or spirit of the disclosure. One skilled in the art will recognize that the disclosed features may be used singularly, in any combination, or omitted based on the requirements and specifications of a given application or design. When an implementation refers to “comprising” certain features, it is to be understood that the implementations can alternatively “consist of” or “consist essentially of” any one or more of the features. Other implementations will be apparent to those skilled in the art from consideration of the specification and practice of the disclosure.

It is noted in particular that where a range of values is provided in this specification, each value between the upper and lower limits of that range is also specifically disclosed. The upper and lower limits of these smaller ranges may independently be included or excluded in the range as well. The singular forms “a,” “an,” and “the” include plural referents unless the context clearly dictates otherwise. It is intended that the specification and examples be considered as exemplary in nature and that variations that do not depart from the essence of the disclosure fall within the scope of the disclosure. Further, all of the references cited in this disclosure including patents, published applications, and non-patent literature are each individually incorporated by reference herein in their entireties and as such are intended to provide an efficient way of supplementing the enabling disclosure as well as provide background detailing the level of ordinary skill in the art.

1. A system for treatment planning, comprising:
a database of a number of subject profiles, each comprising a QTc or QTc range resulting from or expected to result from administration of a drug to the subject of the profile at a selected amount, optionally wherein the subject profiles are simulated subject profiles obtained from clinical trial simulations on a PK/PD model for the selected drug and/or are actual subject profiles comprising data obtained from actual subjects;
a processor capable of executing computer-executable instructions for determining an expected QTc or QTc range for a current actual subject, wherein the determining is based on the subject profiles and a Baseline QTc and Creatinine Clearance, Weight, Age, Sex, and/or Heart Rate of the current actual subject, which expected QTc or QTc range would be expected to result after administration of the drug to the current actual subject at the selected amount;
optionally comprising a display for visually displaying one or more of the subject's Baseline QTc, a threshold QTc (or allowable % change), an expected median QTc or QTc range expected to result from administration of the drug to the current actual subject at the selected amount, and/or a 95% confidence interval of the expected QTc or QTc range.

2. A treatment management application configured to perform a set of operations comprising:
(i) reading, prompting input of and/or accepting the following as inputs:
a subject's Baseline QTc and one or more of the subject's Creatinine Clearance, Weight, Age, Sex, and/or Heart Rate, optionally before administration of a drug to the subject;
an amount of an oral or IV target dosage of the drug for the subject; and
(ii) executing computer executable instructions to derive from a set of rules an expected QTc or QTc range for the subject based on profiles of other subjects and the subject's Baseline QTc and Creatinine Clearance, Weight, Age, Sex, and/or Heart Rate, which expected QTc or QTc range would be expected to result from administration of the drug to the subject at the selected amount;
(iii) outputting the expected QTc or QTc range, optionally by way of a visual display of one or more of the subject's Baseline QTc, a threshold QTc (or allowable % change), an expected median QTc or QTc range expected to result from administration of the drug to the subject at the selected amount, and/or a 95% confidence interval of the expected QTc or QTc range.

3. The treatment management application of claim 2, wherein the profiles of other subjects comprise a simulated QTc or QTc range expected to result after administration of the drug to the subject of a simulation at the selected amount, optionally wherein the profiles of the other subjects are simulated subject profiles obtained from clinical trial simulations on a PK/PD model for the selected drug and/or are actual subject profiles comprising data obtained from other actual subjects.

4. A method of administering an antiarrhythmic drug to a patient, comprising:
providing patient-related inputs into a treatment management application comprising at least i) a patient's Baseline QTc and ii) one or more of a patient's Creatinine Clearance, Weight, Age, Sex, or Heart Rate;
the treatment management application providing as an output one or more QTc or QTc range for the patient expected from administration of a 1-5 hour IV dose, alone or followed by one or more oral doses, of an antiarrhythmic drug to the patient; and
in response to the one or more QTc or QTc range being within an acceptable range, administering the 1-5 hour IV dose of the antiarrhythmic drug to the patient, optionally followed by administering one or more of the oral doses of the antiarrhythmic drug to the patient.

5. The method of claim 4, wherein the antiarrhythmic drug is selected from sotalol, dofetilide, amiodarone, ibutilide, dronedarone, procainamide, flecainide, or propafenone.

6. The method of claim 5, wherein the antiarrhythmic drug is sotalol hydrochloride.

7. The method of claim 4, wherein the administering comprises administering to the patient:
a 1-hour IV loading dose of sotalol hydrochloride; and
one or more oral doses of 80 mg, 120 mg, 160 mg or 240 mg sotalol hydrochloride.

8. The method of claim 7, wherein:
at least the patient's Baseline QTc and Creatinine Clearance are the patient-related inputs and a target oral dose for the patient is input into the treatment management application; and
the amount and duration of the 1-5 hour IV dose are determined from a set of rules based on at least the patient's creatinine clearance and the target oral dose for the patient.

9. The method of claim 4, wherein:
the treatment management application comprises or is in operable communication with a database comprising a number of simulated and/or actual subject profiles, each of the subject profiles comprising a QTc or QTc range resulting from or expected to result from administration of the antiarrhythmic drug to the subject of the profile at a selected amount and duration;
the output of the one or more QTc or QTc range expected for the patient is derived from a set of rules based on the simulated and/or actual subject profiles and the patient-related inputs.

10. The method of claim 8, wherein:
the treatment management application comprises or is in operable communication with a database comprising a number of simulated and/or actual subject profiles, each of the subject profiles comprising a QTc or QTc range resulting from or expected to result from administration of the antiarrhythmic drug to the subject of the profile at a selected amount and duration;
the output of the one or more QTc or QTc range expected for the patient is derived from a set of rules based on the simulated and/or actual subject profiles and the patient-related inputs.

11. The method of claim 4, wherein the treatment management application comprises or is in operable communication with a neural network trained on an initial set of simulated subject profiles and/or additional simulated and/or actual subject profiles, optionally updated in response to obtaining additional simulated subject profiles and/or actual subject profiles.

12. The method of claim 8, wherein the treatment management application comprises or is in operable communication with a neural network trained on an initial set of simulated subject profiles and/or additional simulated and/or actual subject profiles, optionally updated in response to obtaining additional simulated subject profiles and/or actual subject profiles.

13. The method of claim 4, wherein the output of the one or more QTc or QTc range expected for the patient is determined:
performing stochastic simulations;
using an object-oriented approach for model simulation in which each dose is represented by its closed form exponential representation; and/or
using a plasma curve constructed by superposition; and/or
using real-time Monte Carlo simulations.

14. The method of claim 8, wherein the output of the one or more QTc or QTc range expected for the patient is determined:
performing stochastic simulations;
using an object-oriented approach for model simulation in which each dose is represented by its closed form exponential representation; and/or
using a plasma curve constructed by superposition; and/or
using real-time Monte Carlo simulations.

15. The method of claim 4, wherein the administering comprises administering to the patient:
a 1-hour IV loading dose of dofetilide; and
one or more oral doses of 125 μg, 250 μg, or 500 μg dofetilide.

16. The method of claim 15, wherein:
at least the patient's Baseline QTc and Creatinine Clearance are the patient-related inputs and a target oral dose for the patient is input into the treatment management application; and
the amount and duration of the 1-5 hour IV dose are determined from a set of rules based on at least the patient's creatinine clearance and the target oral dose for the patient.

17. The method of claim 16, wherein:
the treatment management application comprises or is in operable communication with a database comprising a number of simulated and/or actual subject profiles, each of the subject profiles comprising a QTc or QTc range resulting from or expected to result from administration of the antiarrhythmic drug to the subject of the profile at a selected amount and duration;
the output of the one or more QTc or QTc range expected for the patient is derived from a set of rules based on the simulated and/or actual subject profiles and the patient-related inputs.

18. The method of claim 16, wherein the treatment management application comprises or is in operable communication with a neural network trained on an initial set of simulated subject profiles and/or additional simulated and/or actual subject profiles, optionally updated in response to obtaining additional simulated subject profiles and/or actual subject profiles.

19. The method of claim 16, wherein the output of the one or more QTc or QTc range expected for the patient is determined:
performing stochastic simulations;
using an object-oriented approach for model simulation in which each dose is represented by its closed form exponential representation; and/or
using a plasma curve constructed by superposition; and/or
using real-time Monte Carlo simulations.

20. A method, comprising:
performing clinical trial simulations on a PK/PD model for a selected drug to obtain a number of simulated subject profiles, each comprising a simulated QTc range expected to result after administration of the drug at a selected amount to the subject of the simulation.
